(19) United States
(12) Patent Application Publication
(10) Pub. No.: US 2010/0292798 A1
(43) Pub. Date: Nov. 18, 2010

(54) HEMI-PROSTHESIS

(76) Inventor: Gianluca Maestretti, Walenried (CH)

Correspondence Address: STROOCK & STROOCK & LAVAN, LLP, 180 MAIDEN LANE, NEW YORK, NY, 10038, US

(21) Appl. No.: 12/738,707

(22) PCT Filed: Oct. 16, 2008
(86) PCT No.: PCT/US08/80171

§ 371 Date: Apr. 19, 2010

Related U.S. Application Data
(60) Provisional application No. 60/981,341, filed on Oct. 19, 2007.

(51) Int. Cl. A61F 2/44

(52) U.S. Cl. 623/17.12; 623/17.16

(57) ABSTRACT

An intervertebral disc prosthesis (10) for total, partial, or nuclear replacement of an intervertebral disc space includes an anchoring member (20) and a cushioning member (30). The anchoring member is sized and configured to engage an inferior vertebra. The cushioning member is sized and configured to directly contact an endplate of the superior vertebra and to couple to the anchoring member, which is secured to the inferior vertebra. Thus, the prosthesis includes a single endplate for contacting the inferior vertebra and a cushioning member for directly contacting the superior vertebra. In use, the prosthesis facilitates movement between the vertebral bodies by (a) direct articulation via sliding or articulation between the vertebral bodies and the prosthesis, (b) indirect articulation via deformation (e.g., compression) of the prosthesis, or (c) a combination thereof.

### CROSS-REFERNCE TO RELATED APPLICATIONS

This application claims the benefit of U.S. Provisional Application No. 60/981,341, filed on Oct. 19, 2007, entitled “HEMI-PROSTHESIS AS A NUCLEUS OR TOTAL DISC REPLACEMENT,” the contents of which is incorporated in its entirety by reference herein.

### BACKGROUND OF THE INVENTION

Millions of people suffer from back pain, specifically the wearing down of the intervertebral discs. Such pain is generally the result of a pathological condition of an intervertebral disc, caused by, for example, injury or age-related degeneration. Current treatment options for back pain range from conservative bed rest to highly invasive surgical procedures including spinal fusion and discectomy. Spinal fusion, i.e., fusion or immobilization of the vertebrae on each side of the afflicted intervertebral disc, is a procedure that offers pain relief and an increased stability of the fused segment. Discectomy, i.e., surgical removal of part of the intervertebral disc is another surgical option.

Nucleus and/or total disc replacement with a prosthesis is another known option for relief of back pain and a number of such prostheses have been proposed. An intervertebral disc prosthesis for nucleus or total disc replacement preferably restores the normal mobility of the disc, restores the disc height and re-creates healthy disc pressure.

Accordingly, a need exists for an intervertebral disc prosthesis to replace a degenerated disc that provides significant spinal motion while eliminating as much pain as possible.

### SUMMARY OF THE INVENTION

Briefly stated, a preferred embodiment of the present invention is directed to an intervertebral disc prosthesis for total, partial, or nuclear replacement of an intervertebral disc space. The prosthesis includes an anchoring member for engaging an adjacent vertebra, preferably the inferior vertebra, and a cushioning member for directly contacting the other vertebra, preferably the endplate of the superior vertebra. The cushioning member is also preferably securely coupled to the anchoring member. Thus, the prosthesis preferably includes a single endplate for contacting the inferior vertebra and a cushioning member for directly contacting the superior vertebra. In use, the prosthesis facilitates movement between the vertebral bodies by (a) direct articulation via sliding or articulation between the vertebral bodies and the prosthesis, (b) indirect articulation via deformation (e.g., compression) of the prosthesis, or (c) a combination thereof.

In one particularly preferred embodiment, the intervertebral disc prosthesis is sized and configured for implantation between adjacent superior and inferior vertebral bodies. The prosthesis includes an anchoring member and a cushioning member. The anchoring member includes a superior surface and an inferior surface. The anchoring member is secured to the inferior vertebral body when the prosthesis is in an implanted position. The cushioning member is coupled to the anchoring member and is in direct contact with the superior vertebral body when the prosthesis is in the implanted position. The cushioning member preferably articulates with respect to the superior vertebral body and is preferably compressible. The cushioning member preferably includes an outer membrane for coupling to the anchoring member and for contacting the superior vertebral body. The outer membrane preferably surrounds an inner material.

### BRIEF DESCRIPTION OF THE DRAWINGS

The foregoing summary, as well as the following detailed description of the preferred embodiments of the application, will be better understood when read in conjunction with the appended drawings. For the purposes of illustrating the intervertebral disc prostheses of the present application, there are shown in the drawings preferred embodiments. It should be understood, however, that the application is not limited to the precise arrangements and instrumentalities shown. In the drawings:

FIG. 1 illustrates a side elevational, partial cross-sectional view of an intervertebral disc prosthesis according to a first preferred embodiment of the present application implanted between adjacent superior and inferior vertebral bodies;

FIG. 2 illustrates a side elevational, partial cross-sectional view of an intervertebral disc prosthesis according to a second preferred embodiment of the present application implanted between adjacent superior and inferior vertebral bodies;

FIG. 3 illustrates a side elevational, partial cross-sectional view of an intervertebral disc prosthesis according to a third preferred embodiment of the present application implanted between adjacent superior and inferior vertebral bodies;

FIG. 4A illustrates a top perspective view of an intervertebral disc prosthesis according to a fourth preferred embodiment of the present application;

FIG. 4B illustrate with a side elevational, partial cross-sectional view of the intervertebral disc prosthesis shown in FIG. 4A and implanted between adjacent superior and inferior vertebral bodies; and

FIG. 5 illustrates a side elevational, partial cross-sectional view of an intervertebral disc prosthesis according to a fifth preferred embodiment of the present application implanted between adjacent superior and inferior vertebral bodies.

### DETAILED DESCRIPTION OF THE INVENTION

Certain terminology is used in the following description for convenience only and is not limiting. The words “right”, “left”, “top” and “bottom” designate directions in the drawings to which reference is made. The words “inwardly” and “outwardly” refer to directions toward and away from, respectively, the geometric center of the device and designated parts thereof. The words, “anterior”, “posterior”, “superior”, “inferior” and related words and/or phrases designate preferred positions and orientations in the human body to which reference is made and are not meant to be limiting. The terminology includes the above-listed words, derivatives thereof and words of similar import.

Certain exemplary embodiments of the invention will now be described with reference to the drawings. In general, such embodiments relate to an intervertebral disc prosthesis. More specifically, the present application is directed to an intervertebral disc prosthesis for total, partial, or nuclear replacement of a spinal disc. The prosthesis of the present application will be described in connection with spinal disc replacement herein, but one of ordinary skill in the art will understand that the system as well as the components thereof may be used for replacement of tissue in other parts of the body including, for example, knee, hip, shoulder, finger or other joint replacement.

As will be described in greater detail below, the intervertebral disc prostheses 10, 10′, 10″, 10′″, 10″″ of the preferred embodiments preferably include at least one cushioning member 50, 50″″ sized and configured to (a) contact an endplate of a first vertebra V and (b) couple to an anchoring member 20, 20′, 20″, 20′″ secured to a second vertebra. That is, the intervertebral disc prostheses 10, 10′, 10″, 10′″, 10″″ include a single endplate for contacting one adjacent vertebra, preferably the inferior vertebra V₁, and a cushioning member 50, 50″″ for directly contacting the other vertebra, preferably the superior vertebra V_{S}.

The intervertebral disc prostheses 10, 10′, 10″, 10′″, 10″″ may, for example, fill the entire intervertebral disc space to replace the entire intervertebral disc space. Alternatively, although a single intervertebral disc prosthesis 10, 10′, 10″, 10′″, 10″″ is shown and described, a plurality of intervertebral disc prosthesis 10, 10′, 10″, 10′″, 10″″ may be used to fill the intervertebral disc space. For example, two or more smaller prosthesis 10, 10′, 10″, 10′″, 10″″ may be used to fill the intervertebral disc space. Alternatively, the intervertebral disc prosthesis 10, 10′, 10″, 10′″, 10″″ may be sized and configured to only partially replace an intervertebral disk space, such as for example, to replace a nucleus.

In use, the intervertebral disc prostheses 10, 10′, 10″, 10′″, 10″″ facilitate movement between the vertebral bodies V_{I}, V_{S} by, for example, (a) direct articulation via sliding or articulation between the vertebrae bodies V_{I}, V_{S} and the intervertebral disc prosthesis 10, 10′, 10″, 10′″, 10″″, (b) indirect articulation via deformation (e.g., compression) of the intervertebral disc prosthesis 10, 10′, 10″, 10′″, 10″″, or (c) a combination thereof.

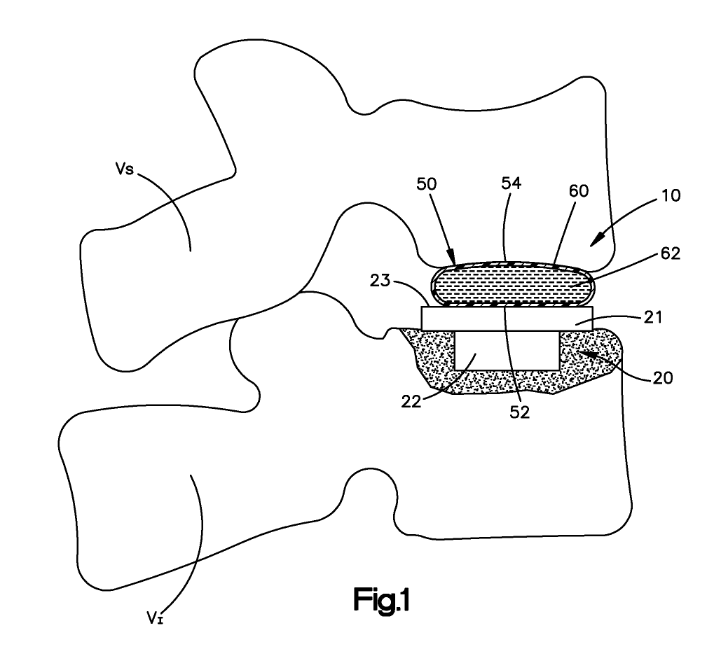

Referring to FIGS. 1-5, the intervertebral disc prostheses 10, 10′, 10″, 10′″, 10″″ of the preferred embodiments include an anchoring member 20, 20′, 20″, 20′″ and a cushioning member 50, 50″″. The anchoring member 20, 20′, 20″, 20′″ is preferably sized and configured to engage one of the adjacent vertebra V, preferably the inferior vertebra V_{I}. The anchoring member 20, 20′, 20″, 20′″ may be securely fixed to the vertebra V, preferably the inferior vertebra V_{I}, in order to absorb the anticipated loads and to hold the prosthesis 10, 10′, 10″, 10′″, 10″″ in place. The anchoring member 20, 20′, 20″, 20′″ may be secured to the vertebra V, preferably the inferior vertebra V_{I}, by any means, including but not limited to: mechanical means including but not limited to screws, keels, teeth, spikes, rivets, nails, blades, clamps, or a combination thereof; chemical means including but not limited to gluing; thermal bonding such as welding, etc. The anchoring members 20, 20′, 20″, 20′″ of the preferred embodiments of the intervertebral disc prostheses 10, 10′, 10″, 10′″, 10″″ of the present application are described herein as being secured to the inferior vertebra V_{I} for convenience only but are not so limited and may be secured to the superior vertebra V_{S} without significantly impacting the design and/or operation of the preferred intervertebral disc prostheses 10, 10′, 10″, 10′″, 10″″. Secondary fixation of the anchoring member 20, 20′, 20″, 20′″ may be achieved by modifying the surface of the anchoring member 20, 20′, 20″, 20′″ to permit bony in-growth. This can be done by any method including, but not limited to hydroxyl apatite, Ti-VPS coating, osteo-conductive materials, etc.

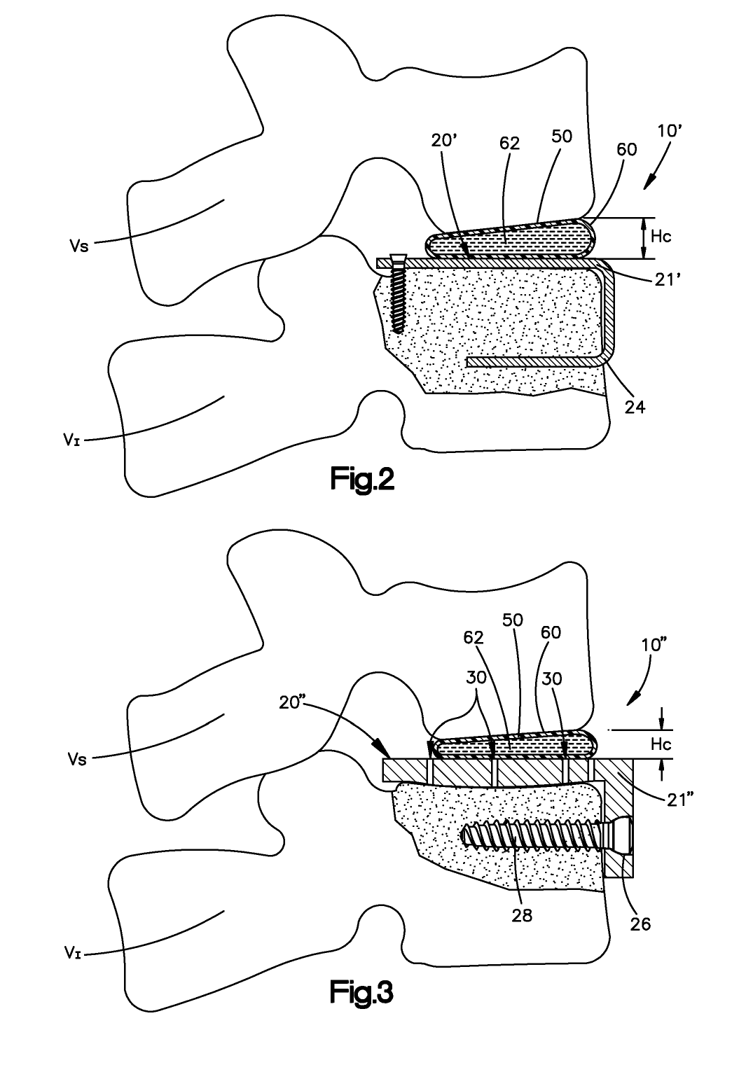

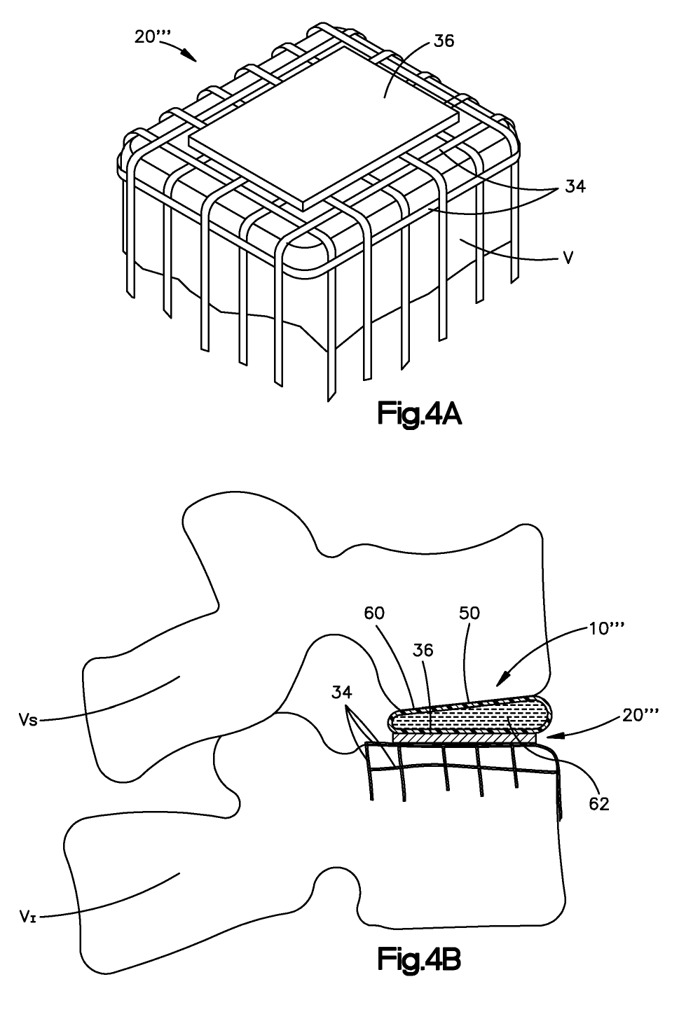

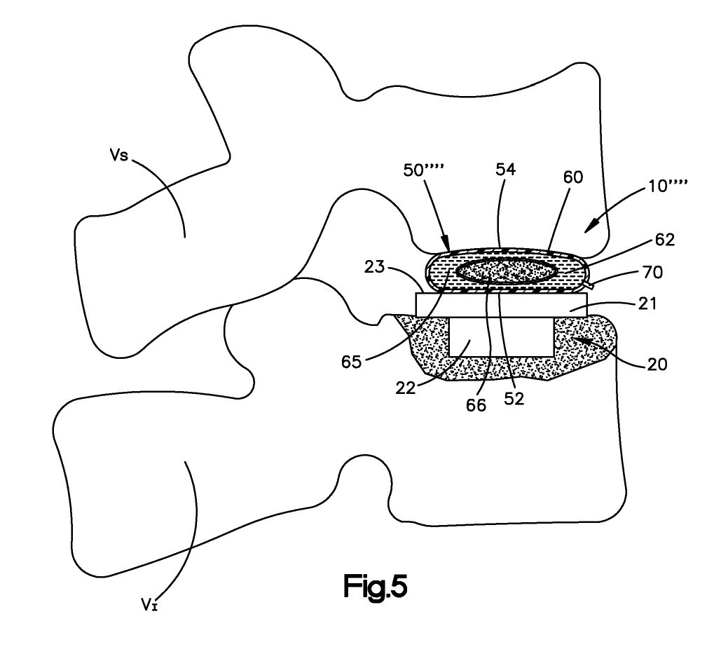

For example, as best shown in FIGS. 1 and 5, the anchoring member 20 of the first and fifth preferred embodiments of the intervertebral disc prosthesis 10, 10″″ may be in the form of a plate 21 including a keel or longitudinal support 22 for engaging the vertebra V, preferably the inferior vertebra V_{I}. Alternatively, in a second preferred embodiment as best shown in FIG. 2, the anchoring member 20′ of the intervertebral disc prosthesis 10′ may be in the form of a plate 21′ including a bone anchoring portion 24 for engaging the vertebra V, preferably the inferior vertebra V_{I}. The bone anchoring portion 24 is preferably sized and configured to be driven into the inferior vertebra V_{I} from an anterior side of the inferior vertebra V_{I}. Alternatively, in a third preferred embodiment as best shown in FIG. 3, the anchoring member 20″ of the intervertebral disc prosthesis 10″ may be in the form of a plate 21″ including a screw hole 26 for receiving a bone screw 28 for engaging the inferior vertebra V_{I}. Alternatively, the screw 28 may be attached to a rod or an external fixation device, as will be apparent to one having ordinary skill in the art. The anchoring member 20″ may also include one or more openings 30 for facilitating bone in-growth and promoting secondary fixation of the anchoring member 20″ to the inferior vertebra V_{I}. Although the openings 30 are shown and described in connection with FIG. 3, the openings 30 may be used in combination with any anchoring member 20, 20′, 20″, 20′″ of the preferred embodiments in order to provide additional affixation of the anchoring member 20, 20′, 20″, 20′″ to the vertebra V, preferably the inferior vertebra V_{I}. Additionally, in a fourth preferred embodiment as best shown in FIGS. 4A and 4B, the anchoring member 20′″ of the intervertebral disc prosthesis 10′″ may be in the form of a mesh 34 coupled to a plate 36. The mesh 34 preferably accommodates bone in-growth and/or screws, staples, nails, pins, etc. for securing the anchoring member 20′″ to the vertebra V, preferably the inferior vertebra V_{I}.

The anchoring member 20, 20′, 20″, 20′″ may be either rigid or incorporate some flexibility and may be made from any biocompatible material including, but not limited to, metals such as CoCr, Ti, Ti alloy, etc. or polymers such as PEEK, PEKK, etc.

The cushioning member 50, 50″″ preferably is sized and configured to (a) engage the anchoring member 20, 20′, 20″, 20′″, (b) contact and/or articulate with the endplate of an adjacent vertebra V, and (c) compress, flex and/or articulate to enable the prosthesis 10, 10′, 10″, 10′″, 10″″ to move (e.g. compress, articulate, translate, etc.) in order to restore the degenerative disc to its original or natural state, as best as possible. That is, the cushioning member 50, 50″″ is preferably constructed to react to loads from the patient's spine and to the provide motion between the superior and inferior vertebra V_{S}, V_{I} in a similar manner to an intervertebral disc that is preferably removed from between the superior and inferior vertebra V_{S}, V_{I} prior to implantation of the anchoring member 20, 20′, 20″, 20′″.

As best shown in FIGS. 1 and 5, a superior surface 23 of the anchoring member 20 of the first and fifth preferred embodiments is coupled to an inferior surface 52 of the cushioning member 50, 50″″ while the superior surface 54 of the cushioning member 50, 50″″ is sized and configured to directly contact and/or articulate with the endplate of the superior vertebra V_{S}. The cushioning member 50, 50″″ may be configured to compress and/or deform (to various extents) as a result of forces acting upon it, or as a result of material being introduced into it. Alternatively the cushioning member 50, 50″″ may be configured to fixedly hold its original shape. In addition, the cushioning member 50, 50″″ may be customized to have variable elasticities based upon the level in the spine where the intervertebral disc prosthesis 10, 10″″ is implanted. For example, if the anchoring member 20 is designed for implantation in the lumbar spine, the cushioning member 50, 50″″ may have a comparatively rigid configuration and if the anchoring member 20 is designed for implantation in the cervical spine, the cushioning member 50, 50″″ may have a comparatively elastic configuration.

The cushioning member 50, 50″″ may include an outer rigid compound or membrane 60 for coupling to the anchoring member 20, 20′, 20″, 20′″ and for contacting and/or articulating with the adjacent, preferably superior vertebra V_{S}, and an inner, softer material or compound 62, located preferably within the outer rigid compound or membrane 60, for permitting compression, flexibility and/or mobility.

For example, the cushioning member 50, 50″″ may be constructed such that the outer membrane 60 is a pillow-like compound or polymeric membrane, filled with a liquid or gel, relatively soft compound 62. The outer membrane 60 may be elastic or non-elastic. Furthermore, as best shown in FIG. 5, the intervertebral disc prostheses 10″″ may include a valve 70 that permits intra-operative filling or deflation of the cushioning member 60 so that the liquid or gel compound 62 may be injected into the outer membrane 60 after implantation. Alternatively, the liquid or gel compound 62 may be injected into the outer membrane 60 prior to implantation. Alternatively or in addition, the amount of filling material liquid or gel compound 62 in the cushioning member 50, 50″″ may be variable, depending upon patient differences, surgeon preferences, implantation location (e.g., lumbar, thoracic or cervical region), etc. Although the valve 70 is shown and described in connection with FIG. 5, the valve 70 may be used in combination with any cushioning member 50 of the preferred embodiments in order to enable the liquid or gel compound 62 to be injected into the outer membrane 60.

The outer membrane 60 may be constructed of a flexible balloon filled with the liquid or gel compound 62 that is in the form of a fluid, a gel, a soft polymer, a solid polymer, etc. Alternatively, the cushioning member 50, 50″″ may be constructed of, for example, a mechanical structure of relatively low stiffness made of metals, polymers, or a combination of these options.

Alternatively, the outer membrane 60 may be comprised of a cushioning membrane filled with the liquid or gel compound 62 that is comprised of an incompressible fluid. Alternatively, the outer membrane 60 may be, for example, an outer balloon that is filled with the liquid or gel compound 62 that is comprised of a solid nucleus and a surrounding fluid or vise versa. The materials that make up the liquid or gel compound 62 and outer membrane 60 may be any of those that were discussed above, or other materials that satisfy the function of pain reduction and increased mobility for the person receiving the prosthesis 10, 10′, 10″, 10′″, 10″″.

The cushioning member 50, 50″″ is preferably permanently, securely fixed to the anchoring member 20, 20′, 20″, 20′″. The cushioning member 50, 50″″ may be fixed to the anchoring member 20, 20′, 20″, 20′″ by any means now or hereafter known including, but not limited to, mechanical bonding, chemical bonding, thermal bonding, sewing, clamping, mechanical interdigitation such as dovetail or tongue in groove, interference fit, etc. or any combination thereof. For example, the cushioning member 50, 50″″ may be fixedly secured to the anchoring member 20, 20′, 20″, 20′″ by gluing, by crimping part of the anchoring member 20, 20′, 20″, 20′″ over part of the cushioning member 50, 50″″, by securing a screw or other mechanical member to the anchoring member 20, 20′, 20″, 20′″ and then embedding said screw or other mechanical member within the cushioning member 50, 50″″, by molding part of the cushioning member 50, 50″″ around part of the anchoring member 20, 20′, 20″, 20′″, etc.

In use, the intervertebral disc prosthesis 10, 10′, 10″, 10′″, 10″″ may be adapted in various ways to fit various needs. For example, as best shown in FIG. 5, the cushioning member 50″″ may be adapted to have multiple sections 65, 66 with any of the sections 65, 66, but preferably the outer section 65, filled with various materials including but not limited to fluids or solid bodies such as balls, cylindrical objects, triangles, etc. The cushioning member 50″″ may be adapted to have various elastic ranges in certain areas, for example, the cushioning member 50″″ may include one or more sections which may be filled with different materials or the various sections may be filled to different pressures.

Alternatively or in addition, the cushioning member 50, 50″″ may include intraoperative possibilities to fill or partially deflate the cushioning member 50, 50″″ thereby providing the surgeon with the ability to control the size and cushioning effect of the cushioning member 50, 50″″. An adaptable cushioning member 50, 50″″ may be arranged such that the size and/or cushioning effect is controllable prior or subsequent to implantation. The cushioning member 50, 50″″ may include various geometries of solid materials such as metals, synthetics, etc. The cushioning member 50, 50″″ may also incorporate osmotic properties and have an osmotic filling, for example, an inner salt concentration.

A height H_{C} of the cushioning member 50, 50″″ may also be adjustable, for example, by changing the pressure of the liquid or gel compound 62 within the outer membrane 60. In addition, the height H_{C} of the cushioning member 50, 50″″ may be variable between the anterior side and posterior side of the cushioning member 50, 50″″ or may also be variable laterally to accommodate the curvature of the spine and the natural angular differences between adjacent endplates of the superior and inferior vertebrae V_{S}, V_{I}. However, the general elasticity of the cushioning member 50, 50″″ and the preferred direct engagement between the cushioning member 50, 50″″ and the vertebra V may permit the patient's superior and inferior vertebrae V_{S}, V_{I} to naturally settle into their preferred orientations and positions relative to each other following a surgery to implant the intervertebral disc prosthesis 10, 10′, 10″, 10′″, 10″″ without specifically forming the cushioning member 50, 50″″ to have a variable height H_{C}.

The intervertebral disc prostheses 10, 10′, 10″, 10′″, 10″″ of the preferred embodiments may be surgically implanted from any angle, including, but not limited to posterior (unilateral or bilateral), transforaminal (unilateral or bilateral), extraforaminal (unilateral or bilateral), extreme lateral (e.g. trans-psoas), anterior, anterior oblique or anterior-lateral.

The implantation may be done by any method but is preferably performed via a minimally invasive technique, for example, through a cannula having, for example, a cannula (not shown) or incision having a maximum diameter or width of about 10 mm to 15 mm.

Subsequent to implantation, the intervertebral disc prostheses 110, 10′, 10″, 10′″, 10″″ of the preferred embodiments preferably have a variable, self-positioning center of rotation.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. An intervertebral disc prosthesis for implantation between adjacent first and second vertebral bodies, the prosthesis comprising:
a generally rigid anchoring member having a superior surface and an inferior surface, the anchoring member secured to the first vertebral body when the prosthesis is in an implanted position; and
a cushioning member coupled to the anchoring member, the cushioning member in direct contact with the second vertebral body when the prosthesis is in the implanted position.

2. The prosthesis of claim 1, wherein the cushioning member is compressible to permit articulation with respect to the second vertebral body.

3. The prosthesis of claim 1, wherein the anchoring member is secured to the first vertebral body by a securing member, the securing member selected from the group consisting of a bone screw, a keel, a spike, a nail, and a bone anchoring portion.

4. The prosthesis of claim 1, wherein the anchoring member includes one or more openings extending from the superior surface to the inferior surface for permitting bone in-growth for secondary fixation of the anchoring member.

5. The prosthesis of claim 1, wherein the anchoring member includes a mesh structure coupled to a plate.

6. The prosthesis of claim 1, wherein the cushioning member includes an outer membrane for coupling to the anchoring member and for contacting the second vertebral body, the outer membrane surrounding an inner material.

7. The prosthesis of claim 6, wherein the inner material is a liquid.

8. The prosthesis of claim 7, wherein the liquid is injected into the outer membrane prior to implantation of the prosthesis.

9. The prosthesis of claim 6, wherein the cushioning member is coupled to the anchoring member by one of mechanical bonding, chemical bonding, thermal bonding, sewing, clamping, mechanical interdigitation and any combination thereof.

10. The prosthesis of claim 6, wherein the outer membrane includes a plurality of sections, at least one of the sections being filled with a different inner material or pressure.

11. The prosthesis of claim 6, wherein the prosthesis includes a valve that permits intra-operative filling or deflation the cushioning member.

12. An intervertebral disc prosthesis for implantation between adjacent first and second vertebral bodies, the prosthesis comprising:
a generally rigid anchoring member having a superior surface and an inferior surface, the anchoring member secured to the first vertebral body when the prosthesis is in an implanted position; and
a cushioning member coupled to the anchoring member, the cushioning member in direct contact with the second vertebral body when the prosthesis is in the implanted position, the cushioning member including an outer membrane surrounding a liquid.

13. The prosthesis of claim 12, wherein the outer membrane is a balloon filled with the liquid.

14. The prosthesis of claim 13, wherein the liquid is compressible.

15. The prosthesis of claim 13, wherein the balloon is coupled to the anchoring member by one of mechanical bonding, chemical bonding, thermal bonding, sewing, clamping, mechanical interdigitation and any combination thereof.

16. The prosthesis of claim 13, wherein the liquid is injected into the outer membrane prior to implantation of the prosthesis via a valve.
